(19) United States
(12) Patent Application Publication
(10) Pub. No.: US 2024/0156934 A1
(43) Pub. Date: May 16, 2024

(54) Compositions And Methods Of MAIT Cell Activation

(71) Applicant: ImmunityBio, Inc., Culver City, CA (US)

(72) Inventor: Patrick Soon-Shiong, Culver City, CA (US)

(73) Assignee: ImmunityBio, Inc., Culver City, CA (US)

(21) Appl. No.: 18/509,777

(22) Filed: Nov. 15, 2023

Related U.S. Application Data
(60) Provisional application No. 63/425,517, filed on Nov. 15, 2022.

(51) Int. Cl. A61K 39/00; A61K 45/06

(52) U.S. Cl. CPC A61K 39/00114; A61K 45/06; A61K 2039/543; A61K 2039/544

(57) ABSTRACT

B cell proliferation and/or memory B cell formation/proliferation can be enhanced by contacting MAIT cells with nogapendekin alfa inbakicept (N-803) to produce stimulated MAIT cells that in turn stimulate B cell proliferation and/or memory B cell formation/proliferation. Such stimulation may be performed in the presence of an antigen or antigen presenting cell. In especially contemplated embodiments, MAIT cell stimulation is performed in vitro to produce immune stimulating compositions and vaccines, or in vivo to enhance an immune response in airway tissues. Most typically, in vivo immune stimulation will be performed by inhalation or intranasal delivery of a composition comprising N-803 and optionally a vaccine component.

This application claims priority to US Provisional Patent application with the Ser. No. 63/425,517, filed Nov. 15, 2022, the contents of which is incorporated by reference in its entirety.

### FIELD OF THE INVENTION

The field of the invention is composition and methods for immune stimulation, and especially MAIT cell-mediated immune stimulation that has enhanced B cell proliferation and/or memory B cell formation.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Mucosal-associated invariant T (MATT) cells are innate-like T cells defined by their semi-invariant αβ T cell receptor (TCR) and are remarkably abundant in human tissues, typically making up 1-4% of all T cells in peripheral blood and up to 10% of airway T cells and 20-40% of liver T cells. The semi-invariant αβ T cell receptor recognizes small-molecule reaction products of bacterial riboflavin synthesis (that represent unique pathway elements in microbial organisms relative to many eukaryotic organisms) that are presented on the major histocompatibility complex (MHC)-related protein-1 (MR1). Thus, the MAIT cell TCR provides an innate capacity to respond to a specific set of potential pathogen-derived ligands without the need for antigen-specific expansion as otherwise required for conventional T cells.

Similar to conventional T cell signaling, MR1-TCR signaling alone is insufficient to fully activate MAIT cells, which also require co-stimulation by CD28, TLR agonists, bacterial products or cytokines. Such cytokines include interleukin (IL)-7, tumor necrosis factor (TNF), type-I interferons (IFNs), IL-1b and/or IL-23. MAIT cells express several cytokine receptors including IL-7R, IL-12R, IL-15R, IL-18R, and IL-23R. IL-7 are thought to enhance MAIT cell responses to bacteria and promotes cytotoxicity. IL-12 and IL-18 are thought to potentiate MR1-dependent bacterial MAIT cell activation. In addition, agonists of various pathogen recognition receptors TLR1, TLR2, and TLR6 in humans, and TLR3, TLR4, TLR6/2, and TLR9 in mice promote MAIT cell activation in an indirect manner through the activation of antigen presenting cells via enhancement of MR1 presentation, stimulation of cytotoxic molecules and inflammatory cytokines or upregulation of co-stimulatory ligands.

Notably, in the absence of TCR-mediated antigen recognition, MAIT cells can also be partially activated by cytokines, such as IL-7, IL-12, IL-15, IL-18, and type-I IFNs, broadening the potential range of pathogens to which MAIT cells can respond to, and exemplary pathogens include various viruses. Unlike bacterial pathogens, viruses lack metabolic pathways to activate MAIT cells through MR1 but activate them instead in an IL-18-dependent manner, together with IL-12, IL-15, and other pro-inflammatory cytokines such as IFNα/β. TCR-independent activation is typically dominated by IFN-γ, perforin, Fas ligand, and granzyme B secretion/release. MAIT cells also secrete selected cytokines, such as IL-17A and IL-22.

Interestingly, and besides a cytotoxic response against pathogens and pathogen-infected cells, a human study had also shown an association between MAIT cells and a pathogen-specific antibody response. For example, supernatants from bacteria- or cytokine-stimulated MAIT cells, when added to purified autologous B cells, were shown to increase the frequencies of plasmablasts and promoted IgA, IgG, and IgM production. However, these effects were found to be mostly MR1-dependent. Moreover, the increase in plasmablasts were likely a result of increased differentiation from memory B cells. Furthermore, bacteria-activated MAIT cell supernatant contained multiple cytokines known to stimulate B cells, including IL-6, IL-10, and IL-21. Thus, B-cell stimulation via MAIT cells is believed to require a microbial infection.

Thus, even though various compositions and methods of MAIT cell-mediated immune stimulation are known in the art, all or almost all of them suffer from several drawbacks. Therefore, there remains a need for improved compositions of immune stimulation, and especially MAIT cell-mediated immune stimulation that triggers enhanced B cell proliferation and/or memory B cell formation/proliferation.

### SUMMARY OF THE INVENTION

The inventive subject matter is directed to various compositions and methods of stimulating MAIT cells in vitro or in vivo and enhancing B cell proliferation and/or memory B cell formation/proliferation using stimulated MAIT cells. Advantageously, such stimulation and enhancement can be performed in the context of vaccine administration, and therapeutic or prophylactic treatment targeting one or more antigens.

In one aspect of the inventive subject matter, the inventor contemplates various methods in which a composition comprising nogapendekin alfa inbakicept (N-803) is administered to a subject by intranasal route. Most typically, such methods will be drawn to stimulation of mucosal-associated invariant T (MATT) cell proliferation, stimulation of mucosal-associated invariant T (MATT) cell-mediated B cell proliferation, and/or stimulation of an immune response in respiratory epithelial tissues.

In such methods, it is contemplated that the composition may delivers between 1 and 50 μg/kg of N-803 to the subject, typically by administration using a nasal spray and/or an inhalable composition. Therefore, it is contemplated that the composition will be administered to the nasal epithelium, the bronchial epithelium, and/or the lung of the subject.

In some embodiments, the composition may further comprise an antigen of a pathogen, or an antigen of a tumor. Viewed from a different perspective, the composition may therefore also comprise a vaccine component such as a viral vaccine (e.g., a recombinant adenoviral vaccine). It is still further contemplated that the subject may be diagnosed with an infection or a cancer, or that the subject may have been previously exposed or suspected to be exposed with a pathogen of unknown origin or species.

In a further aspect of the inventive subject matter, the inventor also contemplates a method of stimulating MAIT cell-mediated B cell proliferation in a subject. Such methods will typically include a step of intranasal administration of an immunotherapeutic combination comprising a vaccine, wherein the vaccine comprises a protein antigen or a nucleic acid encoding an antigen, and N-803.

For example, the protein antigen may be a protein antigen of a bacterial or viral pathogen, or the protein antigen may be a cancer associated antigen, a tumor specific antigen, or a patient- and tumor-specific neoantigen. As will be readily appreciated, the vaccine may be formulated as a protein vaccine, a viral vaccine, a DNA vaccine, or an RNA vaccine. Moreover, contemplated vaccines may further include an adjuvant. Thus, the subject may be diagnosed with an infection or a cancer. Preferably, but not necessarily, the administration delivers between 1 and 50 μg/kg of N-803 to the subject.

In yet another aspect of the inventive subject matter, the inventor contemplates a method of stimulating memory B cell proliferation, and such method will typically include a step of isolating from a patient-derived peripheral blood mononuclear cell fraction a) MAIT cells and b) memory B cells; and a further step of adding N-803 to the MAIT cells and the memory B cells.

In some embodiments, at least 10⁶ MAIT cells and at least 10⁶ memory B cells are isolated, and/or it is further contemplated that the MAIT cells and/or the memory B cells may be (further) expanded prior to adding the N-803. As appropriate or desired, the MAIT cells and the memory B cells may be combined before N-803 is added. Moreover, it is contemplated that such methods may also include a step of further stimulating the MAIT cells and/or the memory B cells with a stimulant selected from the group consisting of a cytokine, an anti-CD3 antibody, an anti-CD28 antibody, PMA (phorbol 12-myristate 13-acetate), ConA (concanavalain A), PHA (phytohaemagglutinin), LPS (lipopolysaccharide), PWM (poke weed mitogen), and α-GalCer (alpha-galactosylceramide). In such methods, the MAIT cells and/or the memory B cells are exposed to the N-803 for a period of between 6-24 hours.

Where desired, the MAIT cells and the memory B cells may be placed in a pharmaceutically acceptable carrier suitable for infusion or injection. Additionally, a vaccine or a protein antigen may be added to the carrier. For example, the protein antigen may be a protein antigen of a bacterial or viral pathogen, a cancer associated antigen, a tumor specific antigen, or a patient- and tumor-specific neoantigen. Where a vaccine is added, the vaccine may be a recombinant viral vaccine, a DNA vaccine, or an RNA vaccine.

In a further aspect of the inventive subject matter, the inventor also contemplates a method of preparing an immunotherapeutic composition for administration to a patient in need thereof that includes a step of isolating from a patient-derived peripheral blood mononuclear cell fraction a) mucosal-associated invariant T (MATT) cells and b) memory B cells; a step of treating the combined MAIT cells and the memory B cells with nogapendekin alfa inbakicept (N-803); and a step of formulating the treated MAIT cells and memory B cells for into a composition for administration to the patient.

Typically, but not necessarily, at least 10⁶ MAIT cells and at least 10⁶ memory B cells are isolated, and where needed or desired, the MAIT cells and/or the memory B cells are expanded prior to adding the N-803. As noted above, it is also contemplated that the MAIT cells and the memory B cells can be combined before treating the MAIT cells with N-803. Moreover, contemplated methods may include a further step of stimulating the MAIT cells and/or the memory B cells with a stimulant selected from the group consisting of a cytokine, an anti-CD3 antibody, an anti-CD28 antibody, PMA (phorbol 12-myristate 13-acetate), ConA (concanavalain A), PHA (phytohaemagglutinin), LPS (lipopolysaccharide), PWM (poke weed mitogen), and α-GalCer (alpha-galactosylceramide).

In further embodiments, the MAIT cells and/or the memory B cells are exposed to the N-803 for a period of between 6-24 hours, and/or the composition may be formulated for infusion or injection. Additionally, it is contemplated that the patient may be diagnosed with an infection or a cancer.

In a still further aspect of the inventive subject matter, the inventor also contemplates a method of preparing an immunotherapeutic composition for administration to a patient in need thereof. Most typically, such method will include a step of isolating from a patient-derived peripheral blood mononuclear cell fraction MAIT cells; a step of treating the MAIT cells with N-803; and a further step of formulating the treated MAIT cells into a composition for administration to the patient.

With respect to the number of cells, the possible expansion of cells, manner of treatment and/or stimulation, and formulation, the same considerations as noted above apply.

Additionally, the inventor contemplates in yet another aspect of the inventive subject matter a method of preparing an immunotherapeutic composition for administration to a patient in need thereof. Such method will typically include a step of isolating from a patient-derived peripheral blood mononuclear cell fraction MAIT cells; a step of treating the MAIT cells with N-803 or IL-15, IL-12, and IL-18, or alternatively with a T×M comprised of biologically active portions of IL-15, IL-12, and IL-18; and a further step of formulating the treated MAIT cells for administration to the patient.

In some embodiments, at least 10⁶ MAIT cells are isolated, and/or the MAIT cells may be expanded prior to adding the N-803. Preferably, but not necessarily, the MAIT cells are treated with N-803 or the T×M. Where desired, the MAIT cells may be further treated with a stimulant selected from the group consisting of a cytokine, an anti-CD3 antibody, an anti-CD28 antibody, PMA (phorbol 12-myristate 13-acetate), ConA (concanavalain A), PHA (phytohaemagglutinin), LPS (lipopolysaccharide), PWM (poke weed mitogen), and α-GalCer (alpha-galactosylceramide). As before, it is also contemplated that such compositions may be formulated for infusion or injection.

Various objects, features, aspects, and advantages of the inventive subject matter will become more apparent from the following detailed description of preferred embodiments.

### DETAILED DESCRIPTION

The inventor has now discovered that B cell proliferation and/or memory B cell formation/proliferation can be enhanced using various compositions and methods in which MAIT cells are exposed to N-803, which may be in the presence of an antigen or antigen presenting cell. Notably, thusly stimulated MAIT cells are capable of stimulating B cell proliferation and/or memory B cell formation/proliferation. Advantageously, such MAIT cell stimulation can be performed in vitro to produce immune stimulating compositions and vaccines, but also in vivo in the context of immune stimulation of tissues in the respiratory tract, for example, using inhaled or sprayed (e.g., intranasal) vaccine compositions.

For example, in one contemplated embodiment, MAIT cell proliferation, MAIT cell-mediated B cell proliferation, and/or stimulation of an immune response in respiratory epithelial tissues in a subject can be achieved by intranasal administration of a composition that comprises N-803. In particularly preferred aspects, the composition is administered from a liquid carrier as a nasal spray into one or both nostrils to so deliver a quantity of about 10 μg/kg of N-803 to the subject, typically once daily over a period of between 3 and 14 days. As will be readily appreciated, such intranasal administration can be performed with or without inhalation to so control the reach of the composition within the respiratory tract. The subject in such example will typically be an individual that is diagnosed with or at risk for an infection or a cancer, or a subject that was previously exposed or suspected to have been exposed with a pathogen of unknown origin or species. Notably, the inventor discovered that activation of MAIT cells in the respiratory epithelium may be effective even where the cytokine function is provided in a IgG-derived construct. Such is particularly unexpected as the predominant antibody classes in mucous membranes and respirator epithelia are IgA and IgM. Without wishing to be bound by any theory or hypothesis, it is contemplated that the prolonged stability of IgG-derived constructs such as N-803 or a T×M may contribute to activation in an environment where IL-15 would otherwise not be stable or biologically effective.

However, it should be appreciated that the compositions and methods presented herein may extend to numerous other applications and uses that will benefit of stimulation of MAIT cells in vitro or in vivo, and especially where such MAIT cells will further enhance B cell proliferation and/or memory B cell formation/proliferation. For example, enhanced B cell proliferation and/or memory B cell formation/proliferation may be advantageous in the context of vaccination, and especially vaccination that delivers a vaccine component to the respiratory tract.

Consequently, it should be appreciated that the intranasal administration need not be necessarily limited to administration of N-803, but that the administered composition may include in addition to N-803 also a vaccine component. As will be readily recognized that the specific nature of the vaccine component is not limiting to the inventive subject matter, but that all vaccine compositions are deemed suitable for use herein. For example, suitable vaccine compositions may comprise one or more protein antigens (which may be crude, isolated, purified, or synthetic) against which an immune response is desired. Moreover, it should be recognized that the type of protein antigen may vary considerably, and especially preferred protein antigens will be (derived) from a bacterial or viral pathogen. Among other suitable examples, bacterial antigens include protein antigens from various strains of Pneumococcus, Vibrio, Salmonella, Meningococcus, Bacillus, Mycobacterium, etc. Similarly, contemplated viral antigens include antigens from various strains of influenza viruses, flaviviruses, parvoviruses, coronaviruses, orthopneumoviruses, lentiviruses, etc. Similarly, it should be appreciated that the protein antigen may also be an antigen of a tumor. Therefore, contemplated tumor antigens include cancer associated antigens, cancer-specific antigens, and patient and tumor specific neoantigens.

While in some embodiments the antigen is a protein or peptide antigen, it is also contemplated that the antigen may be encoded in a recombinant nucleic acid such that upon administration of the recombinant nucleic acid a cell of the subject will express the antigen from the recombinant nucleic acid. Therefore, contemplated nucleic acids will include a segment that encodes one or more antigens wherein that segment is under the control of a regulatory sequence to so allow for expression of the antigenic protein. As will be readily appreciated, such recombinant nucleic acids may be configured in a variety of manners, and especially suitable recombinant nucleic acids include recombinant RNA sequences and recombinant DNA sequences, which may be part of an expression vector, linear sequence containing regulatory elements and the segment, or may be part of a viral vector such as an adenoviral vector.

Consequently, where an antigen or vaccine is delivered with N-803 and/or a stimulated MAIT cell, it should be appreciated that the antigen or vaccine will produce an enhanced immunogenic effect, and especially B cell response and memory B cell formation due to the presence of the stimulated MAIT cells (and N-803, where also present). As such, it should be recognized that contemplated methods can also provide for stimulation of a MAIT cell-mediated B cell proliferation and/or memory B cell proliferation in the presence of a vaccine or protein antigen. In such methods, N-803 is being intranasally administered to a subject as already noted above.

With respect to administration of N-803 to a subject, it is typically preferred that the N-803 is administered in a dosage range of between about 1-50 μg/kg, and more typically between 5 and 25 μg/kg. Moreover, it should be noted that while N-803 administration is typically preferred, various alternative or additional stimulating agents could be administered as well. Particularly contemplated stimulating agents include IL-2, IL-12, IL-15, IL-18, and/or IL-21, and/or a T×M comprised of biologically active portions of IL-15, IL-12, IL-18, most preferably at their clinically known and accepted concentrations, routes of administration, and schedule.

Preferred formulation of N-803 will typically be a liquid suitable for intranasal administration using a atomizer or spray, but other formulations suitable for generation of a mist or vapor are also deemed suitable for use herein. Likewise, N-803 may be adsorbed to inhalable solid phase particles to allow for administration of the compound to the lower respiratory tract. As will be recognized intranasal administration of N-803 may therefore be also suitable to prophylactically stimulate an immune response, or to therapeutically stimulate an immune response against unknown pathogens to which a subject is believed or suspected to have been exposed. Thus, and viewed from a different perspective, MAIT cell stimulation can be performed in vivo by administering N-803 (or other or additional stimulant) to the MAIT cells in situ. As MAIT cells are also found in significant quantities in the liver, administration of contemplated compounds and compositions to the liver by injection or targeting the hepatic circulation is also expressly contemplated.

In further contemplated aspects of the inventive subject matter, MAIT cells can also be activated in vitro in the presence of absence of a vaccine or protein antigen, and/or in the presence or absence of B cells and/or memory B cells. For example, immune stimulation may also be achieved in a variety of methods in which MAIT cell and/or B cell activation by stimulated MAIT cells is performed in vitro. In such approach MAIT cells and memory B cells can be isolated from patient-derived peripheral blood mononuclear cell fraction, and nogapendekin alfa inbakicept (N-803) is then added to the MAIT cells and the memory B cells in an amount and for a time sufficient to stimulate memory B cell proliferation. Thus, such methods will be suitable for the preparation of an immunotherapeutic composition for administration to a patient in need thereof. As will be readily appreciated, contemplated immunotherapeutic compositions need not necessarily include B cells (autologous or from the same patient or subject). Consequently, MAIT cells only can be isolated from patient-derived peripheral blood mononuclear cell fraction, and N-803 is then added to the MAIT cells in an amount and for a time sufficient to stimulate the MAIT cells. So treated cells can then be formulated into a composition for administration to a subject.

MAIT cell isolation can be performed in a variety of ways, but preferably isolation is from the PBMC fraction of whole blood of a subject. For example, PBMCs will be isolated by gradient centrifugation with Ficoll-Paque Plus reagents (e.g., GE, 17-1440-02, Chicago, USA), following the manufacturer's protocol. Where desired, an additional step of platelet removal can be added (e.g., using a low-speed centrifugation/wash step). Staining of surface marker antigens will be performed at 4° C. for 15-20 min in the dark, using the following monoclonal fluorochrome conjugated antibodies: Percep5.5-TCRγδ(B1), PE-CD161 (HP-3G10), APC-Cy7-CD3 (HIT3a), PE-Cy7 or FITC-TCRVα7.2 (3C10). All antibodies are commercially available from BioLegend (San Diego, USA). Freshly isolated PBMCs will be analyzed and sorted/isolated by flow cytometry. MAIT cells will be gated as 7-AAD-TCRγδ⁻, CD3⁺, TCRVα7.2^{hi or +}, and CD161^{hi or +}. Further suitable protocols for isolation and characterization of peripheral blood and tissue-infiltrating MAIT cells by using multicolor flow cytometry are described elsewhere (e.g., Methods Mol Biol 2020; 2098:23-38; or Methods Mol Biol 2020; 2111:285-293). So isolated MAIT cells are then cultured, for example, in RPMI-1640 medium (SH30809.1, America) supplemented with 10% heat-inactivated FBS, and placed in a cell incubator at 37° C. and 5% CO₂ to a desired cell density and quantity.

So isolated MAIT cells can then be stimulated by contacting the cells with N-803 at a concentration of between about 0.1 nM and 10 nM. In addition, also IL-12/15/18 T×m as well as other tri-cytokine T×m constructs (e.g., IL-7/15/21, or IL-15/21, or IL-15/18) as described in WO 2020/023713. In less preferred aspects, individual cytokines can be used to stimulate the MAIT cells, and especially contemplated cytokines include IL-2, IL-7, IL-15, IL-18, and IL-21. Alternatively, or additionally, stimulation may also be performed using α-CD3 (an anti-CD3 antibody), α-CD28 (an anti-CD28 antibody) stimulation, PMA (phorbol 12-myristate 13-acetate), ConA (concanavalain A), PHA (phytohaemagglutinin P), LPS (lipopolysaccharide), PWM (poke weed mitogen), α-GalCer (alpha-Galactosylceramide), etc. Stimulation of MAIT cells can be followed by their enhanced capacity to secrete cytokines and/or chemokines that are associated with help for B cell activation, migration, and regulation.

Similarly, B cell isolation will typically follow well-known protocols starting with whole blood or a PBMC fraction of whole blood. For example, PBMCs will be isolated by gradient centrifugation with Ficoll-Paque Plus reagents (e.g., GE, 17-1440-02, Chicago, USA), following the manufacturer's protocol. Where desired, an additional step of platelet removal can be added (e.g., using a low-speed centrifugation/wash step).

Following isolation of PBMCs, B-cells can be isolated by two main approaches: Negative selection, in which B-cells remain “untouched” in their native state. Negative selection techniques are advantageous as it is likely that B-cells remain functionally unaltered by this process. Positive selection, in which B-cells are labelled and actively removed from the sample. There are various commercially available kits that allow for simple and effective negative or positive isolation such as the Miltenyi B-cell Isolation kit II, the Miltenyi Naïve B-cell Isolation kit, or the Miltenyi CD43 Microbeads kit for negative selection, and the Miltenyi CD19 positive Isolation kit for positive isolation. For isolation of memory B cells, positive selection from total B cells or PBMCs using CD27 microbeads can be performed using compositions and methods well known in the art (e.g., using Miltenyi Memory B Cell Isolation Kit).

Once isolated, B cell can be cultured as is well known. For example, B cells are seeded at 1×10⁵ cells/well in 24-well tissue culture plates and cultured using serum-free medium and supplements, such as ImmunoCult™-XF B Cell Base Medium and ImmunoCult™-ACF Human B Cell Expansion Supplement. The cells are passaged every 3-4 days up to 14 days or desired cell density or number. Alternatively, B cells can also be cultivated using feeder cells following known protocol as described elsewhere (J Immunol. 2016 Nov. 15; 197(10): 4163-4176.)

Isolated and stimulated MAIT cells can then be combined with isolated B cells and cultured for a time sufficient to stimulate memory B cell proliferation and/or memory B cell formation/proliferation. Alternatively, it should be noted that culture supernatant from MAIT cell culture after contact with N-803 may also be used in the B cell culture to so activate the B cells via secreted factors from the MAIT cell supernatant.

Co-culture of MAIT cells with B cells in the presence of N803 or other stimulants is preferably performed at cell ratios (MATT cells with B cells) between 100:1 and 1:100, more typically between 50:1 and 1:50, or between 10:1 and 1:10, or between 5:1 and 1:5, or between 3:1 and 1:3. Total cell density during stimulation will generally be at least 100 cells per mL, or at least 1,000 cells per mL, or at least 10⁵ cells per mL, or at least 10⁵ cells per mL, etc. Moreover, suitable length of stimulation will depend at least in part on the particular manner of MAIT cell activation and cell ratios, but will generally be between 10-120 minutes, or between 120 and 480 minutes, or between 480 minutes and 12 hours, or between 12 hours and 24 hours, or between 24-36 hours, or between 36 and 48 hours, or between 2 and 4 days, and even longer.

As will be readily appreciated, B cell activation may also be may performed in the presence of further stimulants, and contemplated stimulants include N-803, T×M stimulants, one or more cytokines, and/or α-CD3 (an anti-CD3 antibody), α-CD28 (an anti-CD28 antibody) stimulation, PMA (phorbol 12-myristate 13-acetate), ConA (concanavalain A), PHA (phytohaemagglutinin P), LPS (lipopolysaccharide), PWM (poke weed mitogen), α-GalCer (alpha-Galactosylceramide), etc.

Once co-cultured, or otherwise activated, the MAIT cells and/or B cells may then be used directly for infusion, or may be washed with a suitable medium and suspended in a liquid carrier that is pharmaceutically acceptable for infusion to a subject. Therefore, the inventor also contemplates various formulations that can be transfused or injected into a subject. Most typically, he cells are prepared as a cell suspension in a pharmaceutical carrier suitable for intravenous administration, which may further include one or more immune stimulants such as cytokines, checkpoint inhibitors, etc.

Contemplated formulations for transfusion or injection may further include various functional agents, and especially preferred agents include antigens against which immunity is desired, such as bacterial antigens, viral antigens, antigens form pro- or eucaryotic pathogen, and also cancer related antigens such as cancer or tumor specific antigens and patient and tumor specific neoantigens. As will be readily appreciated, such antigens may be provided as polypeptides or encoded in a nucleic acid (e.g., recombinant RNA or DNA sequence containing a segment that encodes the antigen(s), and where the encoded antigen sequence is under the control of one or more regulatory elements that allow for expression of the antigen-encoding segment). In still further contemplated aspects, the nucleic acid may be a naked nucleic acid, or encapsulated or otherwise associated with a lipid fraction to so form a nucleic acid based vaccine. Likewise, the recombinant nucleic acid may be part of a viral vector that is commonly used with vaccine compositions such as adenoviral vectors, etc.

Moreover, the inventor also contemplates a further therapeutic composition in which MAIT cells are activated in vitro as described herein, in which B cells are exposed to the activated cells or cell culture supernatant from such activated MAIT cells, and in which the antibodies produced by the B cells are then isolated and used as a therapeutic product. Most preferably, both the MAIT cells and the B cells are freshly isolated from a subject using methods as known and described above. Upon suitable stimulation of the B cells, antibodies can then be harvested form the medium using one or more affinity reagents such as solid phase immobilized protein A or protein G.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1-29. (canceled)

30. A method of preparing an immunotherapeutic composition for administration to a patient in need thereof, the method comprising:
isolating from a patient-derived peripheral blood mononuclear cell fraction a) mucosal-associated invariant T (MAIT) cells and b) memory B cells; and
treating the combined MAIT cells and the memory B cells with nogapendekin alfa inbakicept (N-803); and
formulating the treated MAIT cells and memory B cells for into a composition for administration to the patient.

31. The method of claim 30, wherein at least 10⁶ MAIT cells and at least 10⁶ memory B cells are isolated.

32. The method of claim 30, wherein the MAIT cells and/or the memory B cells are expanded prior to adding the N-803.

33. The method of claim 30, wherein the MAIT cells and the memory B cells are combined before treating the MAIT cells with N-803.

34. The method of claim 30, further comprising a step of further stimulating the MAIT cells and/or the memory B cells with a stimulant selected from the group consisting of a cytokine, an anti-CD3 antibody, an anti-CD28 antibody, PMA (phorbol 12-myristate 13-acetate), ConA (concanavalain A), PHA (phytohaemagglutinin), LPS (lipopolysaccharide), PWM (poke weed mitogen), and α-GalCer (alpha-galactosylceramide).

35. The method of claim 30, wherein the MAIT cells and/or the memory B cells are exposed to the N-803 for a period of between 6-24 hours.

36. The method of claim 30, wherein the composition is formulated for infusion or injection.

37. The method of claim 30, wherein the patient is diagnosed with an infection or a cancer.

38. A method of preparing an immunotherapeutic composition for administration to a patient in need thereof, the method comprising:
isolating from a patient-derived peripheral blood mononuclear cell fraction mucosal-associated invariant T (MAIT) cells;
treating the MAIT cells with nogapendekin alfa inbakicept (N-803); and
formulating the treated MAIT cells into a composition for administration to the patient.

39. The method of claim 38, wherein at least 10⁶ MAIT cells and at least 10⁶ memory B cells are isolated.

40. The method of claim 38, wherein the MAIT cells and/or the memory B cells are expanded prior to adding the N-803.

41. The method of claim 38, wherein the MAIT cells and the memory B cells are combined before N-803 is used for treating the MAIT cells.

42. The method of claim 38, further comprising a step of further stimulating the MAIT cells and/or the memory B cells with a stimulant selected from the group consisting of a cytokine, an anti-CD3 antibody, an anti-CD28 antibody, PMA (phorbol 12-myristate 13-acetate), ConA (concanavalain A), PHA (phytohaemagglutinin), LPS (lipopolysaccharide), PWM (poke weed mitogen), and α-GalCer (alpha-galactosylceramide).

43. The method of claim 38, wherein the MAIT cells and/or the memory B cells are exposed to the N-803 for a period of between 6-24 hours.

44. The method of claim 38, wherein the composition is formulated for infusion or injection.

45. A method of preparing an immunotherapeutic composition for administration to a patient in need thereof, the method comprising:
isolating from a patient-derived peripheral blood mononuclear cell fraction mucosal-associated invariant T (MAIT) cells;
treating the MAIT cells with nogapendekin alfa inbakicept (N-803), IL-12, and IL-18, or a T×M comprised of biologically active portions of IL-15, IL-12, and IL-18; and
formulating the treated MAIT cells for administration to the patient.

46. The method of claim 45, wherein at least 10⁶ MAIT cells are isolated.

47. The method of claim 45, wherein the MAIT cells expanded prior to adding the N-803.

48. The method of claim 45, wherein the MAIT cells are treated with N-803 or the T×M.

49. The method of claim 45, further comprising a step of further stimulating the MAIT cells with a stimulant selected from the group consisting of a cytokine, an anti-CD3 antibody, an anti-CD28 antibody, PMA (phorbol 12-myristate 13-acetate), ConA (concanavalain A), PHA (phytohaemagglutinin), LPS (lipopolysaccharide), PWM (poke weed mitogen), and α-GalCer (alpha-galactosylceramide).

50. The method of claim 45, wherein the MAIT cells are treated for a period of between 6-24 hours.

51. The method of claim 45, wherein the composition is formulated for infusion or injection.
